(19) United States
(12) Patent Application Publication
(10) Pub. No.: US 2024/0279140 A1
(43) Pub. Date: Aug. 22, 2024

(54) METHOD FOR GENERATING ISOBUTENE, CATALYST FOR GENERATING ISOBUTENE, AND ISOBUTENE GENERATION SYSTEM

(71) Applicants: HONDA MOTOR CO., LTD., Tokyo (JP); NATIONAL INSTITUTE OF ADVANCED INDUSTRIAL SCIENCE AND TECHNOLOGY, Tokyo (JP)

(72) Inventors: Shigehiro YOSHIDA, Saitama (JP); Hiroshi OIKAWA, Saitama (JP); Katsuya SHIMURA, Ibaraki (JP); Tadahiro FUJITANI, Ibaraki (JP)

(21) Appl. No.: 18/434,760

(22) Filed: Feb. 6, 2024

(30) Foreign Application Priority Data
Feb. 7, 2023 (JP) 2023-017127

(51) Int. Cl. C07C 5/22

(52) U.S. Cl. CPC C07C 5/222; C07C 2521/04; C07C 2521/12

(57) ABSTRACT

A method for generating isobutene, for isomerizing normal butene to isobutene, in which generation of by-products can be infinitely suppressed, and a high yield of isobutene can be achieved. The method for generating isobutene includes isomerizing normal butene to isobutene, wherein the normal butene is brought into contact with a basic catalyst in isomerizing. A reaction temperature in isomerizing is preferably in a range from 25° C. to 249° C.

This application is based on and claims the benefit of priority from Japanese Patent Application No. 2023-017127, filed on 7 Feb. 2023, the content of which is incorporated herein by reference.

### BACKGROUND OF THE INVENTION

### Field of the Invention

The present invention relates to a method for generating isobutene, a catalyst for generating isobutene, and an isobutene generation system.

### Related Art

Conventionally, efforts to mitigate climate change or reduce its impact have been continuing. To achieve this, research and development on reduction of carbon dioxide is being carried out. For example, a technology is known which collects exhaust gas or carbon dioxide in the atmosphere and electrochemically reduces it to obtain a valuable substance. The above technology is a promising technology that can achieve carbon neutrality.

By electrochemically reducing carbon dioxide, ethylene is generated. When ethylene is subjected to oligomerization reaction in the presence of an oligomerization catalyst, olefin such as 1-butene is generated. Isobutene that is a structural isomer of normal butene including 1-butene is industrially important hydrocarbon and has various applications as a synthetic intermediate. For example, the reaction of isobutene with methanol or ethanol provides MTBE and ETBE used as additives for gasoline. Furthermore, dimerization and alkylation of isobutene provide isooctane to be added to gasoline. Therefore, technology on a catalyst for promoting the isomerization reaction of isomerizing normal butene to isobutene has been proposed (see, for example, Non-Patent Document 1).
Non-Patent Document 1: Journal of Catalysis 167, 273-278 (1997)

### SUMMARY OF THE INVENTION

Non-Patent Document 1 shows a result of obtaining a high yield of isobutene by isomerizing normal butene to isobutene in the presence of a zeolite catalyst such as a FER type zeolite. On the other hand, in the isomerization reaction disclosed in Non-Patent Document 1, by-products having 3 or 5 carbon atoms are generated in a considerable proportion. Therefore, it has been difficult to separate and collect unreactive normal butene after isomerization reaction. Furthermore, even if unreactive normal butene could be separated and collected, due to generation of by-products, the yield of isobutene was not able to have a predetermined yield or more. As mentioned above, in the method for isomerizing normal butene to isobutene, a technology to further improve the yield of isobutene has been demanded.

The present invention has been made in view of the foregoing, and an object of the present invention is to provide a method for generating isobutene that is a method for isomerizing normal butene to isobutene wherein generation of by-products can be infinitely suppressed and a high yield of isobutene can be achieved.

(1) The present invention relates to a method for generating isobutene, the method including isomerizing normal butene to isobutene, wherein in the isomerizing, the normal butene is brought into contact with a basic catalyst.

The invention of (1) can provide a method for generating isobutene in which the generation of by-products can be infinitely suppressed, and a high yield of isobutene can be obtained.

(2) The method for generating isobutene describe in (1), wherein a reaction temperature in the isomerizing is in a range from 25° C. to 249° C.

According to the invention of (2), a high yield of isobutene can be obtained, and cost for isomerizing can be reduced.

(3) The method for generating isobutene described in (1) or (2), wherein the basic catalyst is any one of a solid basic catalyst, or a catalyst in which a basic metal is supported on a solid basic catalyst or a solid acid catalyst, and the solid acid catalyst is zeolite, and the solid basic catalyst is alumina.

The invention of (3) can provide a method for generating isobutene in which generation of by-products can be infinitely suppressed, and a higher yield of isobutene can be obtained.

(4) The method for generating isobutene described in (3), wherein the basic metal is at least one of an alkali metal or an alkaline earth metal.

The invention of (4) can provide a method for generating isobutene in which a higher yield of isobutene can be obtained.

(5) Furthermore, the present invention relates to a catalyst for generating isobutene, promoting an isomerization reaction of isomerizing normal butene to generate isobutene, wherein the catalyst for generating isobutene is any one of a solid basic catalyst, or a catalyst in which a basic metal is supported on a solid basic catalyst or a solid acid catalyst.

The invention of (5) can provide a catalyst for generating isobutene in which generation of by-products can be infinitely suppressed and a higher yield of isobutene can be obtained.

(6) The catalyst for generating isobutene described in (5), wherein the solid acid catalyst is zeolite, and the solid basic catalyst is alumina.

The invention of (6) can provide a catalyst for generating isobutene in which a high yield of isobutene can be obtained.

(7) The catalyst for generating isobutene described in (5) or (6), wherein the basic metal is at least one of an alkali metal or an alkaline earth metal.

The invention of (7) can provide a catalyst for generating isobutene in which a high yield of isobutene can be obtained.

(8) Furthermore, the present invention relates to an isobutene generation system including: an electrolyzer for generating ethylene by electrolyzing carbon dioxide; a dimerizer for generating normal butene by dimerizing the ethylene; and an isomerizer for generating isobutene by isomerizing the normal butene, wherein the isomerizer includes a catalyst with which the normal butene is brought into contact, and the catalyst is a basic catalyst.

The invention of (8) can collect carbon dioxide in exhaust gas or the atmosphere and generate isobutene that is industrially important hydrocarbon, and therefore can contribute to achievement of carbon neutrality. Furthermore, in the isomerization reaction of generating isobutene by isomerizing normal butene to isobutene, generation of by-products can be infinitely suppressed and a high yield of isobutene can be provided.

(9) The isobutene generation system described in (8), further including a hydration reactor, wherein the hydration reactor separates the normal butene from a mixture including the normal butene generated by the isomerizer and the isobutene, and the normal butene separated by the hydration reactor is returned to the isomerizer.

According to the invention of (9), the yield of isobutene in the isomerizer of the isobutene generation system can be made to be a yield of theoretically 100%.

### BRIEF DESCRIPTION OF THE DRAWINGS

FIG. 1 is a block diagram showing a configuration of an isobutene generation system in accordance with an embodiment of the present invention;

FIG. 2 is a graph showing an isobutene yield for each type of catalyst;

FIG. 3 is a graph showing a relationship between a base amount and catalytic activity (isobutene yield);

FIG. 4 is a graph showing a relationship between Na₂O concentration and isobutene yield when Na is added to a solid acid catalyst;

FIG. 5 is a graph showing a relationship between a loading amount of Na₂O and isobutene yield when Na is added to a solid basic catalyst;

FIG. 6 is a graph showing a relationship between a loading amount of MOx and isobutene yield when various types of metals are added to a solid acid catalyst; and

FIG. 7 is a graph showing a relationship between a base amount and catalytic activity (isobutene yield) when various types of metals are added to a solid acid catalyst.

### DETAILED DESCRIPTION OF THE INVENTION

### Catalyst for Generating Isobutene

A catalyst for generating isobutene in accordance with this embodiment is a catalyst for promoting isomerization reaction of isomerizing normal butene to isobutene. In this specification, the normal butene refers to a balanced mixture in which 1-butene, cis-2-butene, and trans-2-butene as structural isomers are present in a balanced state.

The catalyst for generating isobutene in accordance with the embodiment is a basic catalyst. When a basic catalyst is used as the catalyst for generating isobutene, not only preferable isobutene yield is achieved, but also the reaction temperature can be made low, cost required to generate isobutene can be reduced. In this embodiment, the basic catalyst is any one of a solid basic catalyst, or a catalyst in which a basic metal is supported on a solid basic catalyst or a solid acid catalyst.

Examples of the solid basic catalyst include alumina. The alumina is not particularly limited, and examples thereof include alumina having a crystal phase such as α, γ, δ, η, and θ types. The above alumina may be used singly or two or more types may be used in combination. The above alumina may be an activated alumina that has been made porous. Activated alumina can be obtained, for example, by a well-known method of dehydrating hydrates. As the above alumina, a commercial product can be used. Examples of the shape of alumina include granular, powdery, and the like.

Examples of the solid basic catalyst include a metal oxide such as magnesium oxide (MgO) and cerium oxide (CeO₂) in addition to alumina.

The solid acid catalyst on which the basic metal described later is supported can be used as a basic catalyst in accordance with this embodiment. Examples of the solid acid catalyst include zeolite. The zeolite is not particularly limited and examples of the zeolite include FER type zeolite (ferrierite), MFI type zeolite (ZSM-5), MOR type zeolite (mordenite), FAU type zeolite (Y type zeolite), BEA type zeolite (beta type zeolite), CHA type zeolite (chabazite), and the like. The above zeolite may be used singly or two or more types may be used in combination. As the solid acid catalyst, silicon dioxide (SiO₂) can be used other than zeolite.

The solid acid catalyst functions as a basic catalyst when the basic metal is supported on the solid acid catalyst. Examples of basic metals include alkali metals and alkaline earth metals. The alkali metals include Li, Na, K, Rb, Cs, Fr, and the like. The alkaline earth metals include Ca, Sr, Ba, Ra, Be, Mg, and the like. In addition to the above, the basic metal may be Zn, Zr, W, La, and the like. The above basic metal may be supported, for example, in the state of a compound such as oxide or nitrate. The basic metal preferably has a larger period number and a smaller group number in the periodic table, that is, a metal having a higher basicity.

The basic metal may be supported on a solid basic catalyst. The solid basic catalyst functions as a basic catalyst even when the basic metal is not supported, but the function as a basic catalyst can be more improved by supporting the basic metal on the solid basic catalyst.

The method for supporting the basic metal on a solid acid catalyst or a solid basic catalyst is not particularly limited, and can be supported by well-known methods such as impregnation method. The impregnation method is not particularly limited, but, for example, an evaporation to dryness method can be used in which a solid acid catalyst or a solid basic catalyst is impregnated with a solution of a compound of a basic metal such as nitrate, and then dried by warming to evaporate the solvent, followed by calcination. As the impregnation method, an adsorption method, a spray method, or the like, may be used in addition to the above. The basic metal is supported in the pores of a solid acid catalyst or a solid basic catalyst when the catalyst is a porous body. When the solid acid catalyst or the solid basic catalyst is not a porous material, the basic metal may be supported on its outer surface.

The supporting amount of the basic metal is not particularly limited, but can be 0.01 to 10% by mass with respect to the weight of the catalyst when the basic metal is supported on the solid acid catalyst. The above supporting amount is preferably 0.07 to 10% by mass, more preferably 0.1 to 10% by mass, and further preferably 1.0 to 10% by mass. Note here that the supporting amount may be 10% by mass or more.

For the basic catalyst, the basic site determined by CO₂-TPD is preferably 0.1 mmol/g or more, and more preferably 0.2 mmol/g or more. CO₂-TPD can apply a well-known method for determining the amount of the basic site by allowing carbon dioxide (CO₂) as an acid probe molecule to adsorb a basic catalyst and measuring the desorption gas.

### Method for Generating Isobutene

A method for generating isobutene in accordance with this embodiment includes isomerizing in which normal butene as a raw material is brought into contact with the above basic catalyst that is a catalyst for promoting the isomerization reaction. The isomerization reaction of normal butene in the above isomerizing is represented by the following Formula (1).

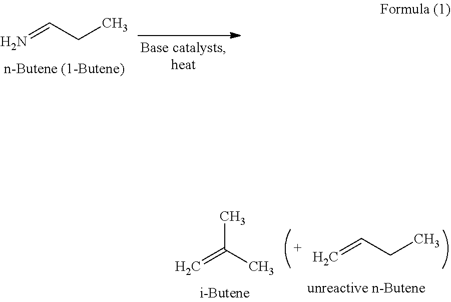

In the above Formula (1), normal butene (n-Butene) is described as 1-butene (1-Butene) for convenience, but the normal butene actually is a balanced mixture in which 1-butene, cis-2-butene, and trans-2-butene are present in a balanced state. The “i-Butene” in the above Formula (1) is isobutene. The “unreactive n-Butene” in the above Formula (1) is unreactive normal butene that is not isomerized by the isomerization reaction and is unreactive. In the above Formula (1), isobutene, and normal butene are described as products, but other than the above products, a small amount of by-products may be generated by the isomerization reaction shown by the above Formula (1).

In isomerizing, the reaction temperature is preferably in a range from 25° C. to 249° ° C. The reaction temperature is more preferably in a range from 25° ° C. to 200° C., further preferably in a range from 25° C. to 150° C., and most preferably in a range from 25° ° C. to 100° C. Use of a basic catalyst in accordance with this embodiment can not only lower the temperature of the isomerizing and reduce the cost required to the isomerizing, but also achieves a more preferable isobutene yield by lowering the reaction temperature.

In isomerizing, gas hourly space velocity (GHSV) of inlet gas including normal butene is preferably in a range from 20000 to 80000 ml·g⁻¹·h⁻¹. Furthermore, the contact time (W/F) is preferably in a range from 0.0008 to 0.0031 g·min·ml⁻¹. Thus, the conversion of normal butene can be improved. The conversion of the normal butene mentioned above is represented by the following Formula (2).

$$Normal butene conversion ( \% ) = ( ( Flow rate of normal butene of reaction gas ) - ( Material amount of normal butene of the inlet of the reactor ) ) / ( Flow rate of normal butene at the outlet of the reactor ) \times 100 \qquad ( 2 )$$

The selectivity of isobutene mentioned above is represented by the following Formula (3), and the yield of isobutene mentioned above is represented by the following Formula (4).

$$Isobutene selectivity ( \% ) = ( Amount of isobutene in generated gas ) / ( Total amount of products ) \times 100 \qquad ( 3 )$$

$$Isobutene yield ( \% ) = ( Isobutene selectivity ( \% ) ) \times ( Normal butene conversion ( \% ) ) \div 100 \qquad ( 4 )$$

The method for generating isobutene in accordance with this embodiment may include any steps other than the above isomerizing. For example, the method may include preparing reaction gas by diluting isobutene with N₂ gas at a predetermined diluting rate. Furthermore, the method may include separating unreactive normal butene from generated gas, and further include reusing the separated normal butene as a reaction gas. Since the method for generating isobutene in accordance with this embodiment can infinitely suppress the generation of by-products, unreactive normal butene can be easily separated from the generated gas. Furthermore, when the normal butene separated from the generated gas is used as a reaction gas, theoretical yield of isobutene can be improved to near 100%.

Separating of unreactive normal butene from the generated gas mentioned above can be achieved by, for example, a hydration reaction (water addition reaction) represented by the following Formula (5).

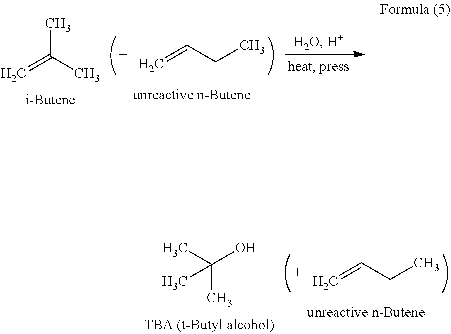

Since only isobutene can be converted to TBA (tert-butyl alcohol) that is a liquid or a solid at room temperature by the hydration reaction in the above Formula (5), normal butene that is gas at room temperature can be easily separated from the generated gas mentioned above. As a specific method, for example, a well-known method such as a method using an aqueous solution including a heteropolyacid having at least one element selected from Mo, W and V as a condensation coordination element and reacting at a temperature of less than 100° C. can be used. Note here that TBA can be effectively used by conversion to isooctane by well-known dimerization techniques, hydrogenation techniques, and the like. This is because isooctane can be used as a base material for gasoline.

### Isobutene Generation System

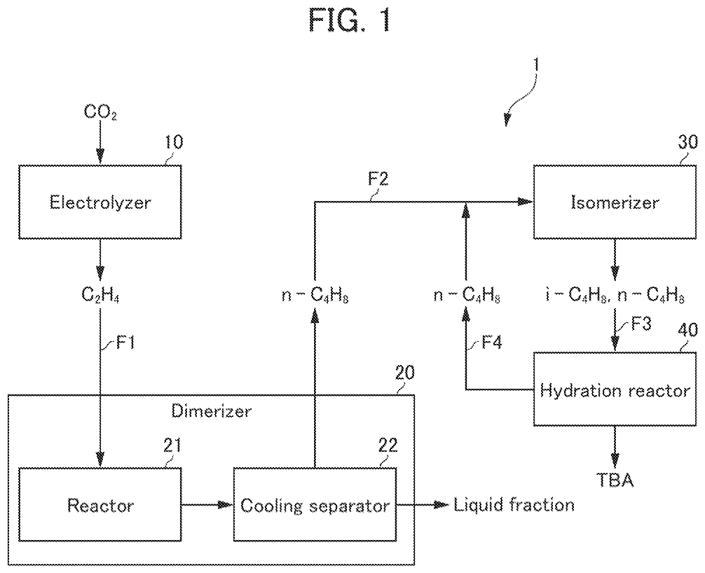

An isobutene generation system 1 in accordance with this embodiment includes, as shown in FIG. 1, an electrolyzer 10, a dimerizer 20, an isomerizer 30, a hydration reactor 40, and flow paths F1 to F4 for connecting them.

The electrolyzer 10 is a device for generating ethylene (C₂H₄) by electrochemically reducing carbon dioxide (CO₂). The electrolyzer 10 reduces carbon dioxide by an electrolytic cell for reducing carbon dioxide. Examples electrolytic cell include an electrolytic cell including at least a cathode and an anode. The cathode electrochemically reduces carbon dioxide to generate hydrocarbon such as ethylene (C₂H₄), and reduces water to generate hydrogen. The anode oxidizes hydroxide ions to generate oxygen. Ethylene (C₂H₄) generated by the electrolyzer 10 is supplied to the dimerizer 20 through the flow path F1.

A supply source of carbon dioxide (CO₂) supplied to the electrolyzer 10 is not particularly limited, and may be separated and collected from the air, or may be separated and collected from exhaust gas discharged from a combustion facility such as a boiler.

The dimerizer 20 is a device for dimerizing ethylene (C₂H₄) supplied through the flow path F1 by a dimerization reaction to generated normal butene (n-C₄H₈). The dimerizer 20 includes a reactor 21, and a cooling separator 22. The dimerizer 20 can generate normal butene (n-C₄H₈) in a yield of, for example, 80% or more.

The reactor 21 carries out an oligomerization reaction of ethylene in the presence of, for example, an olefin oligomerization catalyst to generate an olefin having the number of carbon atoms increased, such as normal butene (n-C₄H₈), 1-hexene or 1-octene. Examples of the olefin oligomerization catalyst include a solid acid catalyst using silica alumina or zeolite as a carrier and a transition metal complex compound. Examples of metal atoms supported on the above carrier include Ni.

The cooling separator 22 carries out gas-liquid separation with respect to generated gas that has been subjected to an oligomerization reaction in the reactor 21. In the olefin having the number of carbon atoms increased included in the generated gas, since the boiling point rises in response to an increase in the carbon number, by setting the temperature of the cooling separator 22 to be not less than the boiling point of normal butene (n-C₄H₈) as the target substance and be less than the boiling point of the other olefins having 6 or more carbon atoms, normal butene (n-C₄H₈) and the other olefins having 6 or more carbon atoms can be easily gas-liquid separated. The normal butene (n-C₄H₈) separated by the cooling separator 22 is supplied to the isomerizer 30 through the flow path F2. The other olefins having 6 or more carbon atoms are separated and discharged as liquid fractions by the cooling separator 22.

The isomerizer 30 is a device for generating isobutene (i-C₄H₈) from normal butene (n-C₄H₈) supplied through the flow path F2. The isomerizer 30 includes the above basic catalyst. The basic catalyst is filled in, for example, a catalyst layer of a fixed bed reactor provided in the isomerizer 30. When gas including normal butene (n-C₄H₈) is allowed to flow through the above catalyst layer, normal butene (n-C₄H₈) and the basic catalyst are brought into contact with each other so that the isomerization reaction is promoted. The isomerizer 30 may include a diluter for diluting normal butene (n-C₄H₈) with N₂ gas at a predetermined diluting rate in addition to the above. Furthermore, well-known devices capable of adjusting the flow amount of the gas including normal butene (n-C₄H₈), as well as temperatures and pressure of the fixed bed reactor may be included. A mixture including isobutene (i-C₄H₈) generated by the isomerizer 30 and unreactive normal butene (n-C₄H₈) is supplied to the hydration reactor 40 through the flow path F3.

The hydration reactor 40 is a device for separating unreactive normal butene (n-C₄H₈) from the mixture including isobutene (i-C₄H₈) and unreactive normal butene (n-C₄H₈) supplied through the flow path F3 by the hydration reaction in the above Formula (5). Only isobutene (i-C₄H₈) is converted into TBA (tert-butyl alcohol) by a hydration reaction by the hydration reactor 40, and separated from the normal butene (n-C₄H₈) by gas-liquid separation. The TBA (tert-butyl alcohol) separated by the hydration reactor 40 is converted into isooctane and the like by the existing technique. The unreactive normal butene (n-C₄H₈) separated by the hydration reactor 40 is returned to the isomerizer 30 by the flow path F4 as the return flow path. The flow path F4 may be connected to the middle of the flow path F2 or may be connected to the isomerizer 30.

The isobutene generation system 1 having the above structure achieves the following effects. Since the isobutene generation system 1 has the isomerizer 30 having a basic catalyst, the isobutene (i-C₄H₈) as the target substance can be obtained in a high yield, and generation of by-products can be infinitely suppressed. Furthermore, the isobutene generation system 1 includes a hydration reactor 40 for separating a mixture including isobutene (i-C₄H₈) generated by the isomerizer 30 and unreactive normal butene (n-C₄H₈), and the flow path F4 as the return flow path for returning unreactive normal butene (n-C₄H₈) separated by the hydration reactor 40 to the isomerizer 30. Thus, the yield of isobutene (i-C₄H₈) with respect to normal butene as raw material substance in the isomerizer 30 can be made be a yield of theoretically 100%.

Furthermore, the isobutene generation system 1 can generate isobutene that is an industrially important hydrocarbon by recovering carbon dioxide in exhaust gas and the atmosphere, and therefore contributes to achievement of carbon neutrality.

In the above, the preferable embodiments of the present invention are described, but the present invention is not limited to the above embodiments of the present invention, and modifications and improvements within a scope that can achieve the object of the present invention are included in the present invention.

In the above embodiments, the isobutene generation system 1 in which devices are directly connected through the flow paths F1 to F4 is described. Embodiments are not limited to the above. Each device may store a product in a storage tank such as a gas cylinder, and may supply a product to other devices by transporting the storage tank.

### EXAMPLES

Hereinafter, the present invention is described in detail with reference to Examples. However, the present invention is not limited to these Examples.

### Relationship Between Type of Basic Catalysts and Isobutene Yield

Using catalysts shown in Table 1 below, reaction gas is allowed to flow at the flow rates of normal butene (n-C₄H₈) of 15 ml/min, and N₂ of 50 ml/min to cause the isomerization reaction. Reaction conditions include a temperature of 200° ° C., a pressure of 0.1 MPa, a reaction time of 40 min, and a catalyst amount of 0.2 g.

TABLE 1

| Catalyst. | |
|---|---|
| No | Details |
| 1 | SiO₂-Al₂O₃ (Al₂O₃ 0.5% SA101, manufactured by Fuji |
| | Silysia Chemical Ltd. ) |
| 2 | SiO₂-Al₂O₃ (Al₂O₃ 1%) |
| 3 | SiO₂-Al₂O₃ (Al₂O₃ 13.8% JRC-SAL-4, manufactured by |
| | JGC Catalysts and Chemicals Ltd. ) |
| 4 | SiO₂-Al₂O₃ (Al₂O₃ 28.6% JRC-SAH-1, manufactured |
| | by, JGC Catalysts and Chemicals Ltd. ) |
| 5 | γ-Al₂O₃ (JRC-ALO-3, manufactured by Sumitomo |
| | Chemical Company Limited) |
| 6 | γ-Al₂O₃ (NKHD-24HD, manufactured by Sumitomo |
| | Chemical Company Limited) |
| 7 | γ-Al₂O₃ (JRC-ALO-9, manufactured by Nippon Light |
| | Metal Company, Ltd. ) |
| 8 | γ-Al₂O₃ (NEOBEAD GB-45, manufactured |
| | by Mizusawa Industrial Chemicals, Ltd. ) |
| 9 | γ-Al₂O₃ (AKP-G15, manufactured by Sumitomo |
| | Chemical Company Limited) |
| 10 | γ-Al₂O₃ (JRC-ALO-6, manufactured by Nikki- |
| | Universal Co., Ltd. ) |
| 11 | θ-Al₂O₃ (JRC-ALO-10, manufactured by Nippon Light |
| | Metal Company, Ltd. ) |
| 12 | Montmorillonite (K10, manufactured by Sigma |
| | Aldrich) |
| 13 | Montmorillonite (K30, manufactured by Sigma |
| | Aldrich) |
| 14 | SiO₂-MgO (MgO 29.1% JRC-SM-2, manufactured by |
| | Mizusawa Industrial Chemicals, Ltd.) |
| 15 | ZrO₂ |
| | (SUR-100, manufactured by Daiichi Kigenso |
| | Kagaku Kogyo Co., Ltd.) |
| 16 | CeO₂ (HS, manufactured by Daiichi Kigenso Kagaku |
| | Kogyo Co., Ltd. ) |
| 17 | MgO (JRC-MGO-4 500A, manufactured by |
| | Ube Material Industries, Ltd. ) |

Note here that the catalyst of Catalyst. No. 2 of Table 1 was prepared by the following procedure. Aluminum nitrate 9 hydrate, TEOS, and urea were used for precipitation by the homogeneous precipitation method (urea 10 equivalent, aging at 80° ° C. for 48 h), and the obtained fine powder was calcined at 700° C. for 6 h to prepare the catalyst.

### Gas Chromatography

The generated gas generated by the isomerization reaction with each catalyst shown in Table 1 was quantitatively analyzed by gas chromatography. Measurement conditions by the gas chromatography are as follows.

### Measurement Conditions

Measuring device: GC-2014 (manufactured by Shimadzu Corporation)
Columns: Rtx-1 (RESTEK, length: 60 m, inner diameter: 0.25 mm, thickness: 0.5 mm)
Carrier gas: N₂ (total flow rate: 50 ml/min, purge flow rate: 3.0 ml/min)
Split ratio: 66.1 (column flow rate: 0.70 ml/min) Injection: 250° C.
Detection: 280° ° C.
Analysis: 10 min at 40° C., then increased to 200° ° C. at 20° C./min and then 9.5 min at 200° ° C. (total 30 min)

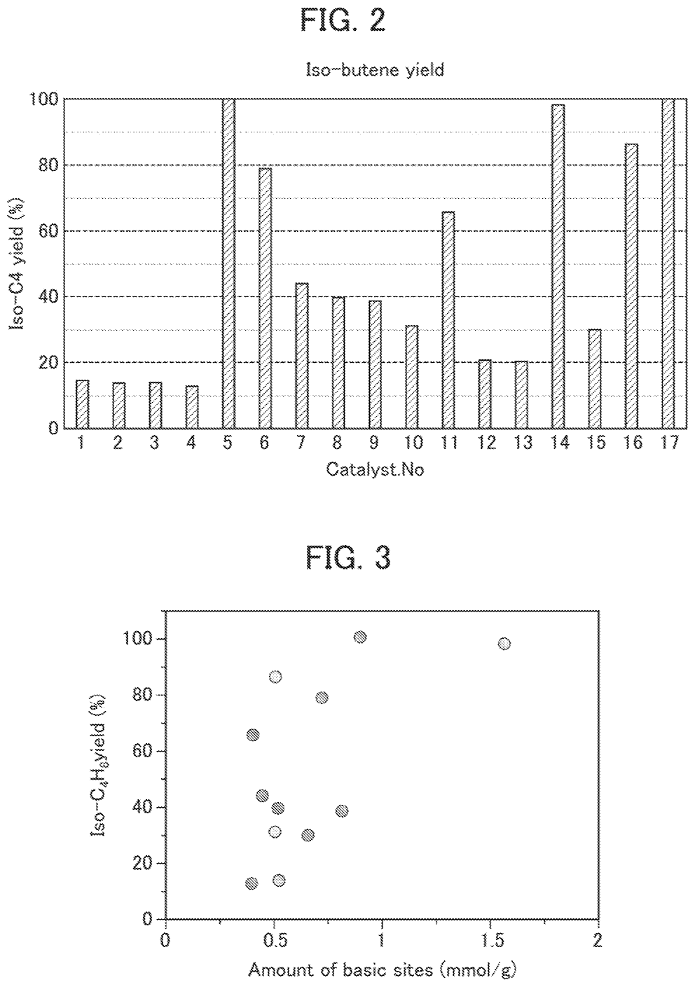

The yield of isobutene was determined based on the analytical results obtained by the above gas chromatography, and the results are shown in Table 2 below and the graph of FIG. 2. Note here that “yield/% iso-C₄H₈” in Table 2 shows the isobutene yield. As shown in Table 2, and FIG. 2, it is clear that catalysts 5, 6, 7, 8, 9, 10, 11, 14, 16, and 17 as the basic catalyst (the number represents Catalyst. Nos. in Table 1, Table 2, and FIG. 2) are used, a high isobutene yield is obtained.

TABLE 2

| | Conversion | | | | | | | | | |
|---|---|---|---|---|---|---|---|---|---|---|
| Catalyst. | rate/% | Yield/% | | | | | | | | C |
| No | 1-C₄H₈ | C₂H₄ | C₃H₆ | iso-C₄H₁₀ | iso-C₄H₈ | 2-C₄H₈ | n-C₄H₁₀ | C5 | C6 | balance |
| 1 | 51.8 | 0.00 | 0.00 | 0.04 | 14.5 | 35.5 | 0.00 | 0.01 | 0.00 | 96.8 |
| 2 | 51.4 | 0.00 | 0.01 | 0.04 | 13.7 | 34.5 | 0.00 | 0.04 | 0.01 | 93.9 |
| 3 | 52.7 | 0.00 | 0.01 | 0.05 | 13.9 | 33.1 | 0.00 | 0.06 | 0.02 | 89.4 |
| 4 | 51.4 | 0.00 | 0.01 | 0.04 | 12.8 | 33.3 | 0.00 | 0.04 | 0.01 | 90.0 |
| 5 | 99.9 | 0.00 | 0.00 | 0.03 | 101.3 | 0.4 | 0.00 | 0.00 | 0.00 | 101.8 |
| 6 | 88.5 | 0.00 | 0.00 | 0.03 | 78.9 | 10.8 | 0.00 | 0.00 | 0.00 | 101.4 |
| 7 | 69.8 | 0.00 | 0.00 | 0.04 | 44.0 | 26.2 | 0.00 | 0.00 | 0.00 | 100.7 |
| 8 | 67.4 | 0.00 | 0.00 | 0.04 | 39.6 | 27.8 | 0.00 | 0.00 | 0.00 | 100.1 |
| 9 | 68.0 | 0.00 | 0.00 | 0.04 | 38.6 | 29.4 | 0.00 | 0.00 | 0.00 | 100.1 |
| 10 | 65.3 | 0.00 | 0.00 | 0.04 | 31.2 | 34.2 | 0.00 | 0.00 | 0.00 | 100.2 |
| 11 | 81.5 | 0.00 | 0.00 | 0.03 | 65.7 | 16.0 | 0.00 | 0.00 | 0.00 | 100.3 |
| 12 | 58.7 | 0.00 | 0.00 | 0.04 | 20.7 | 35.8 | 0.00 | 0.01 | 0.00 | 96.5 |
| 13 | 58.9 | 0.00 | 0.00 | 0.03 | 20.3 | 35.1 | 0.00 | 0.01 | 0.00 | 94.2 |
| 14 | 99.0 | 0.00 | 0.00 | 0.03 | 98.2 | 2.4 | 0.00 | 0.00 | 0.00 | 101.6 |
| 15 | 69.7 | 0.00 | 0.00 | 0.04 | 30.0 | 39.9 | 0.00 | 0.00 | 0.00 | 100.4 |
| 16 | 94.5 | 0.00 | 0.00 | 0.04 | 86.4 | 9.8 | 0.00 | 0.00 | 0.00 | 101.8 |
| 17 | 100.0 | 0.00 | 0.00 | 0.04 | 100.6 | 0.5 | 0.00 | 0.00 | 0.01 | 101.1 |

### Relationship Between Reaction Temperature and Isobutene Yield

The isomerization reaction of normal butene (n-C₄H₈) was carried out using ferrierite (manufactured by Tosoh Co., Ltd., 760HOA, FER (silica/alumina ratio around 60)) and γ-alumina (JRC-ALO-6 manufactured by JGC Universal Co., Ltd.) as catalysts, respectively, at the reaction temperatures shown in Table 3 below. The reaction conditions were the same as those in FIG. 2 except for the reaction temperatures. The results are shown in Table 3. In Table 3, “yield/% iso-C₄H₈” indicates the isobutene yield.

TABLE 3

| | | Reaction | Conversion | | | | | | | | | |
|---|---|---|---|---|---|---|---|---|---|---|---|---|
| | | temperature | rate/% | Yield/% | | | | | | | | C |
| Catalyst | | (° C.) | 1-C₄H₈ | C₂H₄ | C₃H₆ | iso-C₄H₁₀ | iso-C₄H₈ | 2-C₄H₈ | n-C₄H₁₀ | C5 | C6 | balance |
| 760HOA | FER, 58 | 150 | 47.6 | 0 | 0 | 0.04 | 11.8 | 33.1 | 0 | 0 | 0 | 94.5 |
| | | 200 | 49.3 | 0 | 0.01 | 0.05 | 13.2 | 34.7 | 0 | 0 | 0 | 97.3 |
| | | 250 | 53.9 | 0.02 | 0.07 | 0.06 | 18.9 | 34.9 | 0 | 0.04 | 0 | 100 |
| | | 300 | 60.6 | 0.01 | 0.39 | 0.14 | 28.3 | 32 | 0 | 0.29 | 0.04 | 101 |
| | | 350 | 67.3 | 0.03 | 1.23 | 0.28 | 36 | 28.2 | 0.01 | 1 | 0.08 | 99.2 |
| JRC- | γ-Al₂O₃ | 100 | 100 | 0 | 0 | 0.03 | 99.3 | 0.6 | 0 | 0 | 0 | 100 |
| ALO-6 | | 150 | 93.7 | 0 | 0 | 0.04 | 86.5 | 8.6 | 0 | 0 | 0 | 101.5 |
| | | 200 | 65.3 | 0 | 0 | 0.04 | 31.2 | 34.2 | 0 | 0 | 0 | 100.2 |
| | | 250 | 58.6 | 0 | 0 | 0.04 | 22 | 35.4 | 0 | 0 | 0 | 98.1 |

As shown in Table 3, it is clear that γ-alumina as the basic catalyst has a higher isobutene yield than ferrierite, especially in the temperature range less than 250° C., and no by-products are generated. Furthermore, it is clear that the higher the temperature of ferrierite is, the higher the isobutene yield is obtained, while the lower the temperature of γ-alumina as the basic catalyst is, the higher the isobutene yield is obtained.

### Relationship 1 Between Base Amount of Catalyst and Isobutene Yield

The isomerization reaction was carried out using γ-alumina, θ-alumina, silica magnesia, and zeolite with different base amounts, and the relationship between the amount of basic sites and the isobutene yield was determined. The isomerization reaction was measured under the same conditions as in FIG. 2. The amount of basic sites was measured by CO₂-TPD. A catalyst analyzer BELCAT B (manufactured by Microtrac Bell Co., Ltd.) was used as the measuring device. The reaction conditions are as follows. The amount of the catalyst was set to 0.1 g and packed in a quartz cell. As a pretreatment, He (30 ml/min) was allowed to flow and heated at 500° C. for one hour, and then cooled to 50° C. while He was allowed to flow. Then, CO₂ (30 ml/min) was allowed to flow at 50° C. for one hour, CO₂ was adsorbed thereon, He (30 ml/min) was allowed to flow at 50° C. for one hour, and physically adsorbed CO₂ was detached. Thereafter, while He (30 ml/min) was allowed to flow, the temperature was raised from 50° ° C. to 800° C. at 10° C./min, held at 800° ° C. for 30 min, and a desorbed amount of CO₂ was measured to estimate the amount of basic sites. The results are shown in FIG. 3. As shown in FIG. 3, it is clear that as the amounts of basic sites are increased, the isobutene yield tends to be improved.

### Relationship 1 Between Basic Metal Supporting Amount and Isobutene Yield

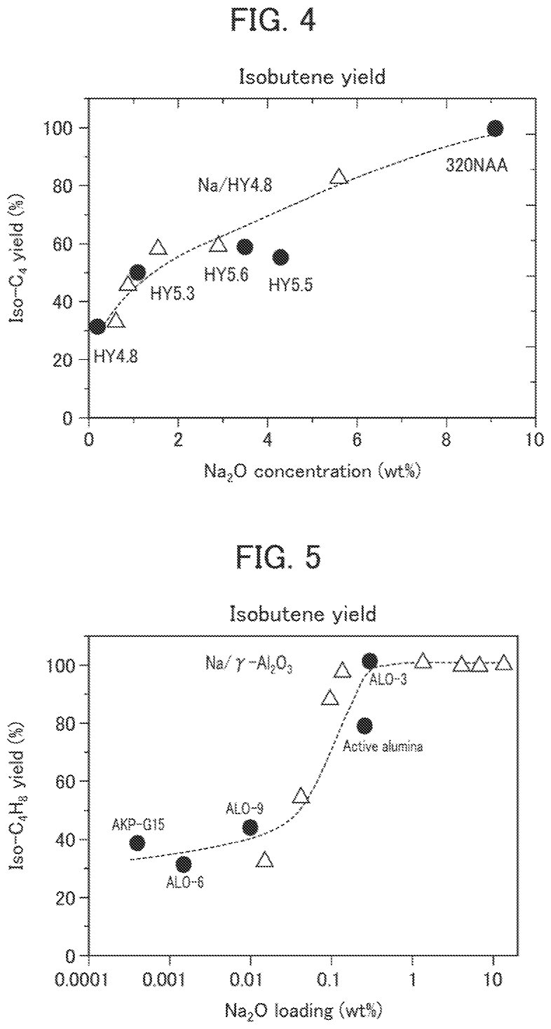

The isomerization reaction was carried out under the same conditions as in FIG. 2 using Y-type zeolite supported with basic metal (Na₂O) (denoted by “O” in FIG. 4), Na-type zeolite “HSZ-320 NAA” (manufactured by Tosoh Co., Ltd.), and a catalyst obtained by adding Na₂O to “JRC—Z-HY 4.8” (manufactured by Catalyst Chemical Industry Co., Ltd.) in Table 4 (denoted by “A” in FIG. 4), and the relationship between the content (% by weight) of Na₂O and the isobutene yield was determined in each catalyst. The results are shown in Table 4 below and FIG. 4.

TABLE 4

| | | | | 1-C₄H₈ | |
|---|---|---|---|---|---|
| | Crystal | SiO₂/ | Na₂O/ | conversion | iso-C₄H₈ |
| Catalyst | structure | Al₂O₃ | wt % | rate/% | yield/% |
| JRC-Z-HY4.8 | Y | 5.2 | 0.2 | 66.5 | 31.3 |
| JRC-Z-HY5.3 | Y | 5.3 | 1.1 | 77.8 | 50.0 |
| JRC-Z-HY5.5 | Y | 5.6 | 4.3 | 83.4 | 58.8 |
| JRC-Z-HY5.6 | Y | 5.6 | 3.5 | 82.1 | 55.2 |
| HSZ-320NAA | Y | 5.5 | 9.1 | 100 | 99.6 |

As shown in Table 4 and FIG. 4, it is clear that in the zeolite that is solid acid catalyst, the more the content of the basic metal (Na₂O) is, the higher the isobutene yield is obtained.

### Relationship 2 Between Basic Metal Supporting Amount and Isobutene Yield

The relationship between the content of Na₂O (% by weight) and the isobutene yield was determined in each catalyst in the same manner as in FIG. 4 for JRC-ALO-6 (manufactured by JGC Universal Co., Ltd.) that is γ-alumina and the catalyst in which different amounts of Na were supported on the JRC-ALO-6 by the impregnation method. Note here that production of catalyst by the impregnation method was carried out by impregnating JRC-ALO-6 with an aqueous solution of NaNO₃, then drying at 110° ° C. for 12 h, and then calcinating at 400° C. for 3 h. The results are shown in Table 5.

TABLE 5

| | Conversion | | | | | | | | | |
|---|---|---|---|---|---|---|---|---|---|---|
| | rate/% | Yield/% | | | | | | | | C |
| Catalyst | 1-C₄H₈ | C₂H₄ | C₃H₆ | iso-C₄H₁₀ | iso-C₄H₈ | 2-C₄H₈ | n-C₄H₁₀ | C5 | C6 | balance |
| Al₂O₃ (ALO-6) | 65.3 | 0.00 | 0.00 | 0.04 | 31.2 | 34.2 | 0.00 | 0.00 | 0.00 | 100.2 |
| Na(0.01%)/Al²O³ | 66.2 | 0.04 | 0.00 | 0.04 | 32.3 | 32.2 | 0.00 | 0.00 | 0.00 | 97.5 |
| Na(0.03%)/Al²O³ | 78.5 | 0.00 | 0.00 | 0.03 | 54.4 | 23.0 | 0.00 | 0.00 | 0.00 | 98.7 |
| Na(0.07%)/Al²O³ | 93.9 | 0.00 | 0.00 | 0.03 | 88.1 | 6.6 | 0.00 | 0.00 | 0.00 | 101.0 |
| Na(0.1%)/Al²O³ | 99.0 | 0.03 | 0.00 | 0.04 | 97.6 | 1.6 | 0.00 | 0.00 | 0.00 | 100.2 |
| Na(1%)/Al²O³ | 100.0 | 0.02 | 0.00 | 0.04 | 100.8 | 0.3 | 0.00 | 0.00 | 0.00 | 101.2 |
| Na(3%)/Al²O³ | 100.0 | 0.00 | 0.00 | 0.03 | 99.7 | 0.7 | 0.00 | 0.00 | 0.00 | 100.4 |
| Na(5%)/Al²O³ | 100.0 | 0.00 | 0.00 | 0.03 | 99.6 | 0.6 | 0.00 | 0.00 | 0.00 | 100.3 |
| Na(10%)/Al²O³ | 100.0 | 0.00 | 0.00 | 0.04 | 100.2 | 0.9 | 0.00 | 0.00 | 0.00 | 101.1 |

### Relationship 3 Between Basic Metal Supporting Amount and Isobutene Yield

The relationship between the content of NazO (% by weight) and the isobutene yield was determined in each catalyst in the same manner as in FIG. 4 for the JRC-ALO-6 (manufactured by JGC Universal Co., Ltd.) (denoted by “O” in FIG. 5) that is γ-alumina and the catalysts in which different amounts of Na were supported on JRC-ALO-6 by the impregnation method (denoted by “A” in FIG. 5). Note here that production of the catalysts by the impregnation method was carried out by impregnating JRC-ALO-6 with an aqueous solution of NaNO₃, then drying at 110° C. for 12 h, and then calcinating at 400° ° C. for 3 h. The results are shown in Table 5. As shown in Table 5 and FIG. 5, in alumina that is solid basic catalyst, it is clear that the more the content of basic metal (Na₂O) is, the higher the isobutene yield is obtained.

### Relationship 1 Between Additive Metal and Isobutene Yield

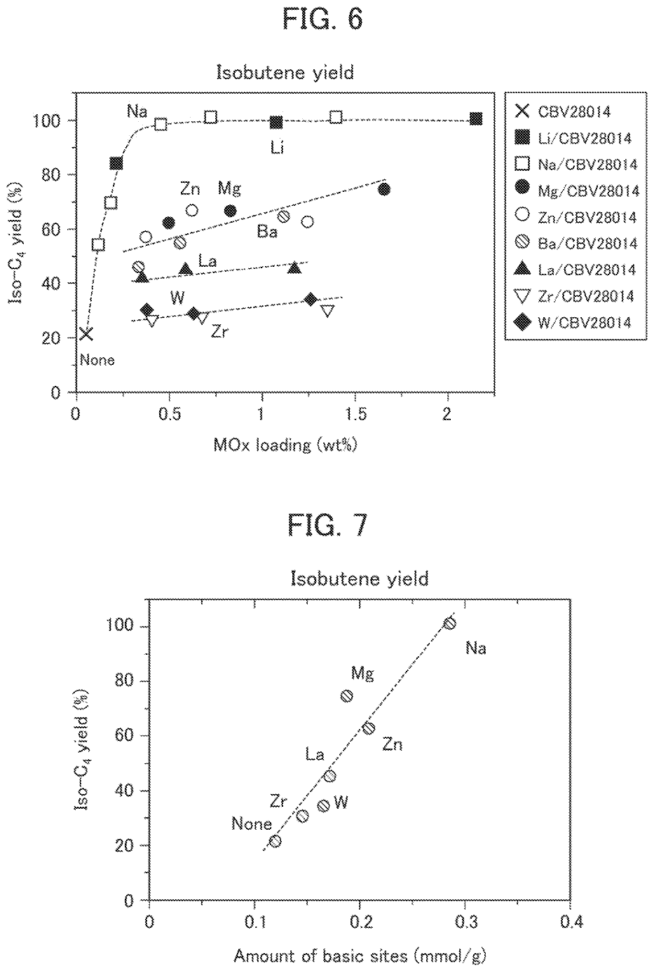

CBV 28014 (manufactured by Zeolyst International, ZSM-5-type, Silica Alumina ratio: 280) that is zeolite was supported with additive metals, Na, Mg, Zn, La, W, and Zr respectively, with an addition amount varied to obtain the relationship between the content of additive metals (MOx equivalent, % by weight) and the isobutene yield in each catalyst in the same manner as FIG. 4. The results are shown in FIG. 6. FIG. 6 clearly shows that the isobutene yield is improved with the increase of the content of the additive metals. Furthermore, as the type of additive metal, Na was the most effective in improving the isobutene yield per added amount, and Mg was the second most effective. Therefore, it is clear that the high isobutene yield can be improved when alkali metal or alkaline earth metal as an additive metal is used.

### Relationship 2 Between Additive Metal and Isobutene Yield

In the same conditions as in FIG. 6, each metal shown in Table 6 as an additive metal was supported on the CBV 28014 with an added amount of 0.7 mol % (equivalent to each metal atom), and the relationship between the type of additive metal and the isobutene yield was determined in the same manner as in FIG. 4. The results are shown in FIG. 6.

TABLE 6

| | Conversion | | | | | | | | | |
|---|---|---|---|---|---|---|---|---|---|---|
| Additive | rate/% | Yield/% | | | | | | | | C |
| metal | 1-C₄H₈ | C₂H₄ | C₃H₆ | iso-C₄H₁₀ | iso-C₄H₈ | 2-C₄H₈ | n-C₄H₁₀ | C5 | C6 | balance |
| None | 54.8 | 0.00 | 0.02 | 0.03 | 21.4 | 33.2 | 0.00 | 0.05 | 0.05 | 100.0 |
| Li | 62.2 | 0.00 | 0.02 | 0.04 | 30.9 | 29.0 | 0.00 | 0.02 | 0.02 | 96.3 |
| Na | 97.1 | 0.00 | 0.00 | 0.03 | 95.9 | 2.9 | 0.00 | 0.00 | 0.00 | 101.8 |
| K | 99.5 | 0.00 | 0.00 | 0.03 | 99.2 | 1.0 | 0.00 | 0.00 | 0.00 | 100.7 |
| Rb | 99.3 | 0.00 | 0.00 | 0.03 | 98.1 | 1.3 | 0.00 | 0.00 | 0.00 | 100.0 |
| Cs | 99.7 | 0.00 | 0.00 | 0.03 | 99.3 | 1.0 | 0.00 | 0.00 | 0.00 | 100.6 |
| Zn | 77.8 | 0.00 | 0.01 | 0.03 | 60.8 | 17.9 | 0.00 | 0.01 | 0.00 | 101.2 |
| Mg | 77.7 | 0.00 | 0.01 | 0.03 | 60.3 | 17.9 | 0.00 | 0.01 | 0.00 | 100.7 |
| Ca | 81.3 | 0.00 | 0.01 | 0.04 | 66.0 | 15.7 | 0.00 | 0.01 | 0.00 | 100.6 |
| Sr | 83.5 | 0.00 | 0.01 | 0.03 | 68.9 | 14.4 | 0.00 | 0.00 | 0.00 | 99.8 |
| Ba | 81.4 | 0.00 | 0.01 | 0.04 | 67.1 | 15.6 | 0.00 | 0.01 | 0.00 | 101.7 |
| Y | 73.2 | 0.00 | 0.01 | 0.04 | 52.5 | 20.4 | 0.00 | 0.01 | 0.00 | 99.7 |
| La | 72.5 | 0.00 | 0.01 | 0.04 | 51.5 | 21.1 | 0.00 | 0.01 | 0.00 | 100.2 |
| Ce | 60.9 | 0.00 | 0.01 | 0.04 | 31.7 | 29.0 | 0.00 | 0.02 | 0.01 | 99.8 |
| Ti | 60.4 | 0.00 | 0.02 | 0.04 | 28.6 | 30.3 | 0.00 | 0.02 | 0.01 | 97.7 |
| Zr | 60.2 | 0.00 | 0.02 | 0.04 | 30.5 | 29.5 | 0.00 | 0.02 | 0.01 | 99.8 |

From the results of Table 6, it is clear that a higher isobutene yield improvement effect can be obtained by using, as an additive metal, a metal having a larger period number and a smaller group number in the periodic table, that is, a metal having a higher basicity.

### Relationship 2 Between Base Amount of Catalyst and Isobutene Yield

The amount of the basic site of each catalyst added with various metals in FIG. 6 was measured by CO₂-TPD in the same manner as in FIG. 3, and the relationship between the amount of the basic site and the isobutene yield was determined. The results are shown in FIG. 7. As shown in FIG. 7, there is a proportional relationship between the amount of basic sites and the yield of isobutene, and it is clear that as the amount of basic sites is increased, the yield of isobutene is improved.

### EXPLANATION OF REFERENCE NUMERALS

1 Isobutene generation system
10 Electrolyzer
20 Dimerizer
30 Isomerizer
40 Hydration reactor

What is claimed is:

1. A method for generating isobutene, the method comprising isomerizing normal butene to isobutene,
wherein in the isomerizing, the normal butene is brought into contact with a basic catalyst.

2. The method for generating isobutene according to claim 1, wherein a reaction temperature in the isomerizing is in a range from 25° C. to 249° C.

3. The method for generating isobutene according to claim 1, wherein the basic catalyst is any one of a solid basic catalyst, or a catalyst in which a basic metal is supported on a solid basic catalyst or a solid acid catalyst, and
the solid acid catalyst is zeolite, and the solid basic catalyst is alumina.

4. The method for generating isobutene according to claim 3, wherein the basic metal is at least one of an alkali metal or an alkaline earth metal.

5. A catalyst for generating isobutene, promoting an isomerization reaction of isomerizing normal butene to generate isobutene,
wherein the catalyst for generating isobutene is any one of a solid basic catalyst, or a catalyst in which a basic metal is supported on a solid basic catalyst or a solid acid catalyst.

6. The catalyst for generating isobutene according to claim 5, wherein the solid acid catalyst is zeolite, and the solid basic catalyst is alumina.

7. The catalyst for generating isobutene according to claim 5, wherein the basic metal is at least one of an alkali metal or an alkaline earth metal.

8. An isobutene generation system, comprising:
an electrolyzer for generating ethylene by electrolyzing carbon dioxide;
a dimerizer for generating normal butene by dimerizing the ethylene; and
an isomerizer for generating isobutene by isomerizing the normal butene,
wherein the isomerizer includes a catalyst with which the normal butene is brought into contact, and
the catalyst is a basic catalyst.

9. The isobutene generation system according to claim 8, further comprising a hydration reactor,
wherein the hydration reactor separates the normal butene from a mixture including the normal butene generated from the isomerizer and the isobutene, and
the normal butene separated by the hydration reactor is returned to the isomerizer.
